(19) United States
(12) Patent Application Publication
(10) Pub. No.: US 2015/0361124 A1
(43) Pub. Date: Dec. 17, 2015

(54) METHOD FOR THE SOLID-PHASE BASED SYNTHESIS OF PHOSPHATE-BRIDGED NUCLEOSIDE CONJUGATES

(71) Applicant: UNIVERSITÄT HAMBURG, Hamburg (DE)

(72) Inventors: Ivo Sarac, Hamburg (DE); Chris Meier, Jork-Ladekop (DE)

(73) Assignee: Universitaet Hamburg, Hamburg (DE)

(21) Appl. No.: 14/761,700

(22) PCT Filed: Jan. 22, 2013
(86) PCT No.: PCT/EP2013/051156

§ 371 Date: Jul. 17, 2015

(51) Int. Cl. C07H 21/04; C07H 19/10

(52) U.S. Cl. CPC C07H 21/04; C07H 19/10

(57) ABSTRACT

A method for producing phosphate-bridged nucleoside conjugates, in particular poly- or oligonucleosides. In the method, an immobilized cyclosaligenyl derivative of a nucleoside, nucleotide, poly- or oligonucleotide, poly- or oligonucleoside, or an analog thereof, is synthesized, and the subsequent reaction with a nucleophile yields the desired phosphate-bridged nucleoside conjugate, which may subsequently be released from the solid phase.

The invention relates to a method for the solid-phase based synthesis of phosphate-bridged nucleoside conjugates, in particular of oligonucleosides or oligonucleotides.

Phosphate-bridged nucleoside conjugates are of great importance in nature. They are not only significantly involved in metabolic-energetic processes, but are present in nearly all biosyntheses as metabolites. Examples for such phosphate-bridged nucleoside conjugates are nucleoside di- and -triphosphates, e.g. the naturally occurring ribo- and deoxyribonucleoside triphosphates (NTP's and dNTP's), oligonucleosides and oligonucleotides, dinucleoside-polyphosphates, NDP sugars or sugar nucleotides, and nucleoside conjugates with peptides etc.

Naturally occurring ribo- and deoxyribonucleoside triphosphates (NTP's and dNTP's), for example, represent basic building blocks for the enzymatically catalyzed RNA and DNA synthesis in vivo and in vitro, while their analogs have an enormous potential as inhibitors in many biological processes (e.g. processes in which DNA polymerases are involved) or as chemotherapeutics. For this reason, there is great interest in a synthetic access to these compounds. However, not only the synthesis of nucleoside triphosphates, but also their isolation is a big problem. Further, nucleoside triphosphates are susceptible for hydrolysis due to their energy-rich anhydride bonds. Their stability depends on both the counter ion and the pH value of the medium (Z. Milewska, H. Panusz, Anal. Biochem. 1974, 57, 8-13). Several methods for the synthesis of nucleoside di- and -triphosphates, including solid-phase based methods, have been described in the prior art (see e.g. Y. Ahmadibeni, K. Parang, Org. Lett. 2005, 7, 5589-5592; Y. Ahmadibeni, K. Parang, J. Org. Chem. 2006, 71, 5837-5839; R. K. Gaur, B. S. Sproat, G. Krupp, Tetrahedron Lett. 1992, 33, 3301-3304; K. Burgess, D. Cook, Syntheses of Nucleoside Triphosphates; Chem. Rev. 2000, 100, 2047-2059; I. Zlatev, T. Lavergne, F, Debart, J.-J. Vasseur, M. Manoharan, F. Morvan, Org. Lett., 2010, 12, 2190-2193; Crauste C, Périgaud C, Peyrottes S., J. Org. Chem. 2009, 74, 9165-9172; I. Zlatev, J. G. Lackey, L. Zhang, A. Dell, K. McRae, S. Shaikh, R. G. Duncan, K. G. Rajeev, M. Manoharan, Bioorganic & Medicinal Chemistry 21 (2013) 722-732).

Oligonucleotides are also of great practical interest, because these compounds have a wide range of applications in e.g. genetic testing, research, and forensics. The comparatively small nucleic acids can be manufactured with a user-specified sequence, and so are very important for the synthesis of artificial gene, the polymerase chain reaction (PCR), for DNA sequencing, library construction and as molecular probes. Oligonucleotides are often synthesized in 3′-5′ direction on a solid-phase using phosphoramidite building blocks derived from protected 2′-deoxynucleosides (dA, dC, dG, and T), ribonucleosides (A, C, G, and U), or chemically modified nucleosides, e.g. locked nucleic acids (LNA) (see e.g. McBride, L J, Caruthers, M H, 1983, Tetrahedron Letters, 24, 245 248; Beaucage, S. L., Iyer, R. P., 1992, Tetrahedron 48, 2223-2311).

To obtain the desired oligonucleotide, the building blocks are sequentially coupled to the growing oligonucleotide chain in the order required by the sequence of the product. The process has been fully automated since the late 1970s and uses the co called solid-phase synthesis approach. Upon the completion of the chain assembly, the product is released from the solid-phase to solution, deprotected, and collected. The occurrence of side reactions sets practical limits for the length of synthetic oligonucleotides (up to about 200 nucleotide residues) because the number of errors accumulates with the length of the oligonucleotide being synthesized. Products are often isolated by high-performance liquid chromatography (HPLC) to obtain the desired oligonucleotides in high purity. Typically, synthetic oligonucleotides are single-stranded DNA or RNA molecules around 15-25 bases in length.

WO 2010/015245 A1 and WO 2010/127666 A1 both disclose methods for the synthesis of phosphate-bridged nucleoside conjugates using so-called cycloSaligenyl (cycloSal) nucleoside phosphate triesters. In the method of WO 2010/127666 A1 a cycloSal nucleoside is bound to a linker and immobilized subsequently via the linker on a solid phase or bound to a linker already bound to the solid-phase. Subsequently a nucleophile is reacted with the immobilized cycloSal nucleoside.

Object of the present invention is to improve the current methods for preparing phosphate-bridged nucleotide bioconjugates, in particular the preparation of oligonucleosides and oligonucleotides.

The object is solved by the method of claim 1. Preferred embodiments are specified in the dependent claims.

It has surprisingly been found that a phosphate-bridged nucleoside conjugate of the general formula

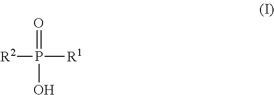

or a salt thereof, wherein R¹ is a nucleoside, nucleotide, polynucleoside, polynucleotide or an analog thereof, and R² is an organic compound or phosphate or pyrophosphate, or a residue thereof, can be obtained in a simple manner in very high yields and purities by a method using a modified “cycloSal” approach. The method according to the present invention comprises the steps of:
a) immobilizing a compound being or comprising R¹ directly or via a linker L on a solid phase SP,
b) coupling to the immobilized compound a substituted or unsubstituted compound of the general formula II

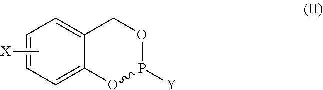

X being H, an electron acceptor or an electron acceptor precursor and Y being halogen, preferably Cl or Br, or —NR³R⁴, wherein R³ and R⁴ are, independently, substituted or unsubstituted alkyl or substituted or unsubstituted aryl, preferably substituted or unsubstituted C₁-C₁₀ alkyl or substituted or unsubstituted C₆-C₂₀ aryl, and wherein the compound II may be substituted one or more times with X,
and oxidizing or sulfurizing the resulting compound to obtain an immobilized compound according to the general formula III

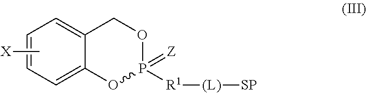

R¹ and X being as defined above, Z being O or S, SP being the solid phase and (L) being the optional linker, and
c) reacting compound III with a nucleophile being or comprising R².

By “phosphate-bridged nucleoside conjugates” is meant herein a compound of the general formula

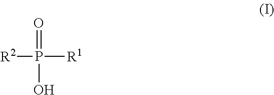

or a salt thereof. R¹ is a nucleoside, nucleotide, polynucleoside, polynucleotide or an analog thereof. The nucleoside, nucleotide, polynucleoside, polynucleotide or an analog thereof is preferably bound to the phosphate atom via an oxygen atom of the sugar component or sugar component analog, in case of a polynucleoside or polynucleotide preferably via an oxygen atom of a terminal sugar component or sugar component analog, e.g. via an oxygen atom at the 2′, 3′ or 5′ C-atom, preferably the 5′ C-atom, of the sugar component, e.g. ribose. R² is any organic compound, preferably a phosphorylated organic compound, or phosphate or pyrophosphate, or a residue thereof. Preferably, R² is a compound or compound residue, or a component analogous to said compound or compound residue, which is present in a living cell, for example an alcohol, a sugar, a steroid, a lipid, a nucleoside, a nucleoside mono-, di- or triphosphate, phosphate or pyrophosphate, or a residue thereof. For such preferred phosphate-bridged nucleoside conjugates are also the term “bioconjugates” is used.

The term “cycloSal nucleotide” or “cycloSaligenyl nucleotide” as used herein means compounds according to the following general formula IV

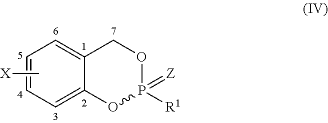

wherein R¹ is a nucleoside, nucleotide, polynucleoside, polynucleotide or an analog thereof, and wherein Z is oxygen (O) or sulfur (S). The term covers cyclic phosphate triester derivatives, in which a salicyl alcohol (saligenol) is diesterified in a cyclic manner with a (mono)phosphate residue, e.g. a phosphate residue bound at the 5□-atom of a ribose or deoxyribose of a nucleoside, nucleotide, polynucleotide, polynucleotide or an analog thereof.

The term “linker” (L) as used herein is understood to mean an organic compound by which another compound, e.g. a compound according to the above formula IV, is covalently bound to a solid phase. A linker usually has at least two functional groups e.g., carboxyl groups —COOH, and is covalently linked with both the compound and the solid phase, thus serving as connecting piece and/or spacer between the compound and the solid phase. Linker compounds are known in the prior art. An example of a linker is a succinyl linker according to formula (V)

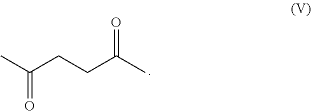

In a chemical formula a linker is represented by the letter “L”. An optional linker is represented by the letter “L” in parentheses, i.e. “(L)”.

An “organic compound” is any compound having bonds of carbon with carbon and with other elements (with the exception of carbon dioxide, carbon monoxide, carbonic acid and its carbonates, and cyanides, isocyanides, cyanates and isocyanates of metals). Examples for organic compounds are carbohydrates, i.e. compounds of carbon and hydrogen, alcohols, aldehydes, ketones, carboxylic acids, amines, amides, nitro compounds, nitriles, alkanethiols, sulfides, sulfates, phosphates, phosphines, metalorganic compounds, aliphatic hydrocarbons, acyclic hydrocarbons, saturated (alkanes), unsaturated (alkenes and alkines), cyclic hydrocarbons, mono- or polycyclic aromatic hydrocarbons (aromatics), heterocycles, biochemical compounds (e.g. amino acids, proteins, nucleosides, nucleotides, hydrocarbons, lipids, steroids) etc. The organic compound may, for example, be a phosphorylated organic compound.

Under the term “carbocycle” cyclic compounds are to be understood of which the ring-forming atoms consist exclusively of C atoms.

A “heterocycle” is a cyclic compound with ring-forming atoms of at least two different chemical elements. In particular, the term means a ring-forming organic component in the ring structure of which at least one carbon atom is replaced by another element, i.e. a heteroatom, for example nitrogen, oxygen, phosphor and/or sulfur. A ring structure can consist of one or more rings connected with each other and may contain one or more identical or different heteroatoms.

The term “nucleophile” as used herein has the usual meaning known by the skilled person. In particular, as used herein, a nucleophile means a molecule containing a negatively polarized region, a negatively polarized functional group or a free electron pair, generally in an energy rich orbital. The term also covers molecules being nucleophile, i.e. relatively electron richer in relation to a reaction partner or to a region of the reaction partner. The reaction partner also is termed electrophile, because it assumes electrons from the nucleophile. Nucleophiles may form covalent bonds by providing electrons to a reaction partner. The electrons necessary for the bond are generally from the nucleophile alone. Nucleophiles can be, and are preferably, negatively charged (anions). Examples for typical nucleophile reagents are carbanions, anions, Lewis bases, aromatics, alcohols, amines, e.g. amino acids, and compounds with olefinic double bonds. The strength of the nucleophilicity depends, for example, on the reaction partner, the basicity, the solvent and sterical factors. The factors affecting the nucleophilicity of a compound are well known to the skilled person, and he can easily determine their nucleophilic properties. The nucleophilicity of a molecule will advantageously be related to the most nucleophilic atom or the most nucleophilic functional group. In case a cycloSal nucleotide according to the above general formula (IV) is employed as an electrophile the electrophilicity of the phosphorus atom can be controlled via the substituent X at the cycloSal aromatic ring (s. C. Meier, J. Renze, C. Ducho, J. Balzarini, Curr. Topics in Med. Chem. 2002, 2, 1111-1121, the disclosure of which is incorporated herein by reference in its entirety). By the introduction of donor substituents at the aromatic ring the electrophilicity can be reduced, acceptor substituents, however, increase the reaction rate of the initial reaction, i.e. the cycloSal ring opening.

An “electron acceptor” is a compound, a region of a compound or a functional group, drawing electrons to it and thereby causing a charge displacement, i.e, a polarization, in a compound. Examples of electron acceptor groups are MeSO₂—, (Me=methyl), —CN, —COOH, ketones or the keto group, formyl, esters or the ester group, —NO₂ and halogens (e.g. F, Cl, Br, I). Preferred esters as electron acceptors are esters whose ester group is situated as close as possible to, preferably directly at, the aromatic ring. Ketones preferred as electron acceptors are ketones whose keto group is situated as close as possible to, preferably directly at, the aromatic ring. An “electron acceptor precursor” is a compound which can be activated, i.e. converted into an electron acceptor, by cleaving off a masking group.

“Esters” are compounds containing the ester group R′—COO—R″, wherein R′ and R″ may be any substituted or unsubstituted, branched- or linear hydrocarbon residues, for example alkyl residues or aryl residues.

“Ketones” are compounds containing the keto group R—CO—R″, wherein R′ and R″ are any substituted or unsubstituted, branched or linear hydrocarbon residues, for example alkyl residues or aryl residues.

By “nucleoside” is meant herein organic molecules consisting of a sugar residue (sugar component) and an organic base (base component), e.g. a heterocyclic organic base, in particular a nitrogen containing heterocyclic organic base, being connected via a glycosidic bond. The sugar residue often is a pentose, e.g. deoxyribose or ribose, but may also be another sugar, e.g. a C₃, C₄ or C₆ sugar. In particular, by nucleoside is meant a compound according to the general formula (VI)

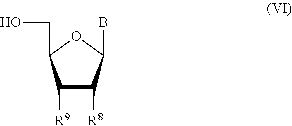

wherein B is a nitrogen containing heterocyclic base, e.g. a nucleobase, and R⁸ and R⁹ are, independent from each other, H or OH. The term also encompasses LNA (locked nucleic acid) nucleosides, i.e. nucleosides, wherein the ribose moiety contains a bridge connecting the 2′ oxygen and 4′ carbon, thereby “locking” the ribose in the 3′-endo (North) conformation.

By “nucleobase” is meant an organic base occurring in RNA and/or DNA. Naturally occurring nucleobases are purines (R) and pyrimidines (Y). Examples for purines are guanine (G) and adenine (A), examples for pyrimidines are cytosine (C), thymine (T) and uracil (U). Phosphorylated nucleoside, for example nucleoside monophosphate (NMP), nucleoside diphosphate (NDP) and nucleoside triphosphate (NTP) are also termed nucleotides. The phosphate, diphosphate (pyrophosphate) or triphosphate group is generally connected with the 5′-C-atom of the sugar component of the nucleoside, but can, for example, also be connected with the 3′-C-atom.

By “nucleoside analog” is meant herein a compound, which naturally does not occur in a living cell of e.g. a human body, but is structurally similar to a nucleoside naturally occurring in a living cell of e.g. the human body in that it contains a sugar component (sugar analog) not naturally occurring in nucleosides of cells or a component analogous to the sugar component of a naturally occurring nucleoside, and a base component (base analog) not naturally occurring in nucleosides of cells or a component analogous to the base component of a nucleoside, such that it can be processed by the cell and/or by viral enzymes essentially analogous to the natural nucleoside, for example phosphorylated and incorporated into an RNA or DNA strand. A sugar analog can, for example, be a carbocycle wherein the ring oxygen atom is replaced by a CH₂ group. Examples for base analogs are 7-deazapurines, isoadenine, hypoxanthine, halogenated pyrimidines (like 5-fluoruracil) etc. A nucleoside analog can itself be a nucleoside. It can, however, also be another compound with the above properties, for example a compound of a heterocyclic base and an acyclic residue and/or a residue that is not a sugar, or a compound of a carbocyclic compound and a sugar residue, or a compound composed of a carbocycle replacing the sugar component, e.g. a modified ribose or deoxyribose, wherein the ring oxygen atom is replaced by a CH₂ group, and a nucleobase (carbocyclic nucleosides). Nucleoside analogs are either itself nucleosides in the above sense or structurally and/or functionally analogous to nucleosides. Since the nucleoside analogs may not necessarily contain a sugar or base component in a narrower sense, it is also spoken of a component analogous to the base component (base analog) or a component analogous to a sugar component (sugar analog). In case a sugar component or a base component is mentioned here the corresponding analogous components of nucleoside analogs shall also be encompassed, unless the context unambiguously requires otherwise. Examples for nucleoside analogs are, for example, AZT (3′-azido-2′,3′-dideoxythimidine, azidothymidine), 2′,3′-dideoxyinosine (didanosine), 2′,3′-dideoxycytidine (zalticabine) and 2-amino-9-((2-hydroxy-ethoxy)methyl)-1H-purine-6(9H)-one (acyclovir). Nucleoside phosphonates can also be nucleoside analogs.

The term “polynucleoside” refers to polymers composed of a sequence of two or more nucleoside units linked by internucleoside bonding groups (“backbone” linkages). The term covers nucleoside polymers wherein the nucleosides are linked by phosphodiester backbone linkages, i.e. polynucleotides, as well as nucleoside polymers linked by structures other than phosphodiester bonds. Such bonds may be modified phosphodiester linkages, e.g. phosphodiester linkages in which one of the non-bridging phosphate oxygens in the linkage is replaced with sulfur, methyl or other atoms or groups, or non-phosphodiester linkages, including phosphorothioate, phosphorodithioate, alkyl- (e.g. methyl-) and arylphosphonate, phosphoramidate, phosphodiester, alkyl- (e.g. methyl-) and arylphosphonothioate, aminoalkylphosphonate, aminoalkylphosphonothioate, phosphorofluoridate, boranophosphate, silyl, formacetal, thioformacetal, morpholino and peptide-based linkages. Chimeric compounds having a mixture of such linkages and/or compounds consisting of or comprising LNA nucleosides are also encompassed by the term “polynucleoside”. The term “polynucleoside analog” refers to a molecule comprising at least one nucleoside analog, the term “oligonucleotide analog” refers to a molecule comprising at least one nucleotide analog.

The term “DNA” or “deoxyribonucleic acid” denotes polynucleotides, wherein the sugar component is deoxyribose. The term in particular comprises polynucleotides wherein the sugar component is deoxyribose, the internucleoside linkages are phosphodiester linkages, and the base components are selected from the group consisting of adenine, cytosine, guanine and thymine.

The term “RNA” or “ribonucleic acid” means denotes polynucleotides, wherein the sugar component is ribose. The term in particular comprises polynucleotides wherein the sugar component is ribose, the internucleoside linkages are phosphodiester linkages, and the base components are selected from the group consisting of adenine, cytosine, guanine and uracil.

The term “oligonucleoside” refers to relatively short polynucleosides. In particular the term refers to molecules consisting of not more than 250 nucleoside units, preferably 2-200, 2-150, or 2-100 nucleosides. The term “oligonucleotide” refers to oligonucleosides wherein the nucleosides are linked by phosphodiester backbone linkages. The term “oligonucleoside analog” refers to a molecule comprising at least one nucleoside analog, the term “oligonucleotide analog” to a molecule comprising at least one nucleotide analog.

By the term “glycosyl phosphate” is meant a phosphorylated glycosyl residue. The glycosyl residue may, for example, be phosphorylated at the C1 atom, but may alternatively or additionally be phosphorylated at other positions, e.g. a C6 atom. A “glycosyl” is a compound with a functional group derived from a sugar by elimination of hemiacetal hydroxyl group. Examples for glycosyl-1-phosphates are: glucose-1-phosphate, mannose-1-phosphate, galactose-1-phosphate, 2-N-acetyl-glucosamine-1-phosphate, 6-deoxygulose-1-phosphate, 2-N-acetyl-galactosamine-1-phosphate, D-fucose-1-phosphate and L-fucose-1-phosphate; each in the α or β configuration at the anomeric center (in case of mannose there is only the α form). An example for glycosyl-6-phosphates is glucose-6-phosphate.

The term “alkyl” as used herein refers to branched or straight-chain (unbranched, linear), saturated or unsaturated, aliphatic (non-aromatic) hydrocarbon groups, for example methyl, ethyl, n-propyl, i-propyl, n-butyl, t-butyl, i-butyl, pentyl, hexyl, heptyl, octyl, nonyl, decyl groups. The term thus encompasses alkenyls and alkynyls. The term also comprises the term “cycloalkyl”, meaning mono-, bi- or polycyclic aliphatic hydrocarbon groups. The term “cycloalkyl” includes, for example, cyclopropyl, methyl-cyclopropyl, 2,2-dimethyl-cyclobutyl, 2-ethyl-cyclopentyl and cyclohexyl. The term “alkyl” also covers the term “heteroalkyl”, being an alkyl wherein at least one carbon atoms is replaced by a “heteroatom”, i.e. a non-carbon atom, e.g. oxygen, sulfur, nitrogen or phosphor. The term “C₁-C₁₀ alkyl” means an alkyl having 1, 2, 3, 4, 5, 6, 7, 8, 9 or 10 C atoms.

The term “aryl” as used herein refers to monocyclic, bicyclic and polycyclic substituted or unsubstituted aromatic hydrocarbons, including a single ring or multiple aromatic rings fused or linked together where at least one part of the fused or linked rings forms the conjugated aromatic system. The aryl groups can typically have from 6 to 20 or more carbon atoms and can include, but are not limited to, e.g. phenyl, naphthyl, biphenyl, anthranyl, tetrahydronaphthyl, phenanthryl, indene, benzonaphthyl, fluorenyl, and carbazolyl. The term also encompasses the term “heteroaryl”, meaning aryls containing at least one heteroatom within the ring structure. The term “C₆-C₂₀ aryl” means an aryl having 6, 7, 8, 9, 10, 11, 12, 13, 14, 15, 16, 17, 18, 19 or 20 C-atoms, including C-atoms of any substituents.

By “amine” is meant compounds of the type R—NH₂, NH—R₂, N—R₃ and N—R₄⁺, R representing a substituted or unsubstituted alkyl or aryl residue, wherein, in case of multiple residues, these can be different or the same. The residues may be closed to a ring, so that the term also encompasses cyclic amines. An amine used as nucleophile has preferably the structure R—NH₂ or NH—R₂.

By the term “protecting group” (PG) as used herein is meant a molecule or molecule residue, which blocks a functional group within a compound during a reaction at another site of the compound and which prevents unwanted (side) reactions. A protecting group can ideally be introduced under the mildest conditions possible, is stable under the subsequent conditions, and can mildly be cleaved off after the reaction. Protecting groups are well known to the skilled person, so that he or she will easily find a suitable protecting group, if necessary, after routine experimentation. Examples for a protecting group are the methyl, acetyl, 2-cyanoethyl, and benzoyl group. An OH group can, for example, be protected by O methylation or O acetylation. OPG, for example, is a protecting group bound to an oxygen atom. Protecting groups are, for example, described in Peter G. M. Wuts, Theodora W. Greene: Green's Protective Groups in Organic Synthesis, 2006, 4th ed., John Wiley & Sons Inc., Hoboken, N.J.

The term “halogen” refers to a group of elements comprising fluorine (F), chlorine (Cl), bromine (Br), and iodine (I). As used herein, the term relates in particular to a halogen residue.

The term “independently” or “independent from each other” as used herein in relation to residues or substituents means that the residues or substituents can be identical or different. In case of a compound according to formula IIb below, for example, the residues R³ and R⁴ may be identical, as in case of formula IIb1, or different. The term “independently for each occurrence” means that residues in the same molecule denoted with the same abbreviation (e.g. “R¹” or “Z”) may be identical or different. In a compound according to formula IIIa with n=3, for example, the four residues “Z” could all be identical, e.g. O, or different, e.g, the monomer having index n=1 could be O, the monomers with indexes n=2 and 3 could both be S, and the Z at the P atom of the cycloSal moiety could again be O.

The method of the invention has a broad applicability to a wide variety of compounds. With the method of the invention any phosphate-bridged nucleoside conjugate can efficiently be prepared. Examples are nucleoside diphosphate glycopyranoses, sugar-nucleoside bioconjugates, nucleoside di- and nucleoside triphosphates, dinucleoside monophosphates, dinucleoside polyphosphates, or nucleoside analogs, which may be employed as “prodrugs”, i.e. precursors of active agents later releasing the active agent. The method of the invention is especially useful for the preparation of poly- or oligonucleosides, in particular RNA, DNA and/or LNA poly- or oligonucleosides, e.g. RNA, DNA and/or LNA poly- or oligonucleotides.

Examples of compounds according to the general formula II below

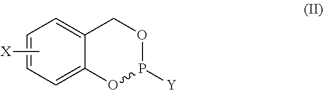

are cycloSaligenyl phosphoramidites according to formula (IIb)

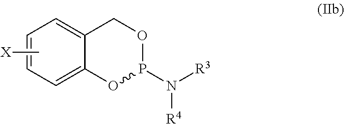

wherein R³ and R⁴ are, independently, substituted or unsubstituted alkyl or aryl, preferably C₁-C₁₀ alkyl or C₆-C₂₀ aryl, e.g. both isopropyl as in formula (IIb1)

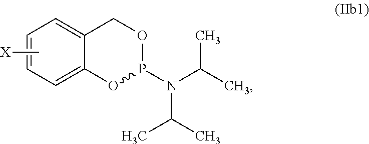

or cycloSaligenyl halogen phosphites according to formula (IIc)

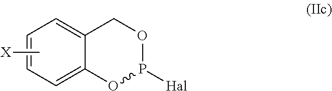

wherein Hal stands for a halogen residue, for example Cl as in formula (IIc1)

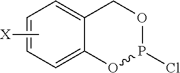

Most preferred halogens Hal are Cl and Br.

The solid phase may, optionally via a linker, for example be bound to an oxygen atom at the 2′- or 3′-C-atom of the sugar component, e.g. a pentose, or sugar component analog of R¹. Other possibilities, however, also exist, e.g. oxygen or nitrogen atoms at other sites of the nucleoside or nucleoside analog. OH groups or, as the case may be, other functional groups at which a chemical reaction is to be avoided can be protected with a protecting group.

In a preferred embodiment of the method of the invention the solid phase or the linker are covalently bound to an oxygen atom of a sugar component of R¹, preferably an oxygen atom bound to a 2′- or 3′ C atom of the sugar component, or to an oxygen atom of a component analogous to a sugar component of R¹, and wherein the residue of formula IIa

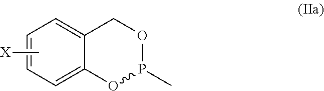

is linked to a different oxygen atom of the same or another sugar component of R¹, preferably an oxygen atom bound to the 5′ C atom of said sugar component, or to a different oxygen atom of the same or another component analogous to a sugar component of R¹. The solid phase or the linker may, for example, be linked to an oxygen atom bound at the 2′- or 3′ C atom of a ribose or deoxyribose of the first nucleoside, i.e. the nucleoside nearest to the solid phase, of an oligonucleoside, and the residue according the above formula IIa may be linked to an oxygen atom of the 5□C atom of the ribose or deoxyribose of the terminal nucleoside, i.e. the nucleoside most remote from the solid phase, of the oligonucleoside.

Especially preferred, compound III is a compound according to formula IIIa

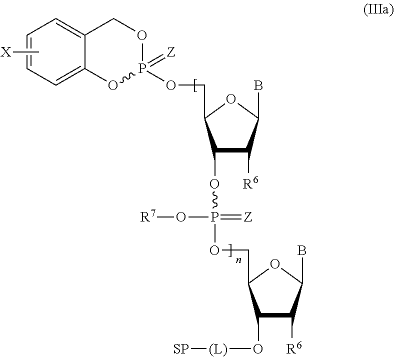

wherein X, SP and (L) are defined as above, Z is, independently for each occurrence, O or S, preferably O, B is, independently for each occurrence, a heterocycle, preferably a nitrogen containing heterocycle, especially preferred a nucleobase, R⁶ is, independently for each occurrence, H, OPG, PG being a protecting group, R⁷ is, independently for each occurrence, H or OPG, PG being a protecting group, and n is an integer≧0. Preferably, B is, independently for each occurrence, one of the nucleobases guanine, adenine, cytosine, thymine or uracil. However, B can, for example, also be a nucleobase analog. Most preferred, B is, independently for each occurrence, one of the nucleobases guanine, adenine, cytosine, thymine or uracil and Z is O or S, preferably O.

R¹ is preferably selected from the group consisting of oligonucleoside, oligonucleotide, oligonucleoside analog, oligonucleotide analog, adenosine, guanosine, cytidine, thymidine, uridine, deoxyadenosine, deoxyguanosine, inosine, deoxycytidine, deoxyuridine, deoxythymidine, 2-thiocytidine, N4-acetyl-cytidine, 2′-O-methyl-cytidine, 3-methyl-cytidine, 5-methyl-cytidine, 2-thiouridine, pseudouridine, dihydrouridine, 5-(carboxyhydroxymethyl)-uridine, 5-carboxymethylaminomethyl-uridine, 5-methylaminomethyl-uridine, 5-methoxy-carbonylmethyl-uridine, 5-methoxy-uridine, ribothymidine, 1-methyl-adenosine, 2-methyl-adenosine, N6-methyl-adenosine, inosine, 1-methyl-inosine, guanosine, N2-dimethyl-guanosine, N2-methyl-guanosine, 7-methyl-guanosine and 2′-O-methylguanosine. Especially preferred R¹ is an oligonucleoside, oligonucleotide, oligonucleoside analog, or oligonucleotide analog.

The solid phase or the linker may alternatively be covalently bound to a nitrogen atom of a base component of the nucleoside, nucleotide, oligonucleoside or oligonucleotide, or to a nitrogen atom of a component analogous to a base component of the nucleoside analog, nucleotide analog, oligonucleoside analog or oligonucleotide analog of R¹.

In preferred embodiments of the method of the invention X, in case of multiple substituents X independently from each other, is selected from the group consisting of H, MeSO₂ (Me=methyl), ketone, formyl, ester, C═O, —COOH, —NO₂, —CN and halogen. In case of a carbonyl group C═O being present in the residue X it is preferred that it is positioned directly at the aromatic ring. The aromatic ring in compound can be one or more times substituted with X, wherein the substituents can be the same or different. The compound according to formula (II) can also be substituted at the C atom 7 (for the numbering see formula IV), for example with methyl, i-propyl, tert-butyl or other alkyl substituents. As the case may be, also the aromatic ring can have further substituents apart from X, for example alkyl or aryl substituents.

In a preferred embodiment of the method of the invention the nucleophile is selected from the group consisting of phosphate, pyrophosphate, glycosyl phosphate, nucleoside, nucleoside monophosphate, nucleoside diphosphate, nucleoside triphosphate, nucleoside analog, nucleoside monophosphate analog, nucleoside diphosphate analog, nucleoside triphosphate analog, α-deprotonated glycosyl, deprotonated mono- or oligosaccharide, amines, amino acids, or salts thereof.

In a further preferred embodiment of the method of the invention the steps a, b and c are repeated. Preferably, in this embodiment, the nucleophile is a nucleoside or nucleoside analog. In this manner oligo- or polynucleosides can be prepared in an advantageous manner. The steps a to c can be repeated until an oligo- or polynucleoside having the desired length or number of monomers, respectively, is received.

The method of the invention preferably comprises the further step(s) of

d) deprotecting compound III and/or cleaving the residue R¹ from the linker or the solid phase. These steps are preferably performed under suitable conditions in order to safely release a compound I produced by the inventive method from the solid phase.

The method of the invention is preferably carried out under an inert gas atmosphere, preferably under nitrogen or argon gas.

The solid phase may be any solid phases, i.e. compounds being essentially insoluble under the conditions chosen. Preferred solid phases are non-swellable or low-swellable materials, e.g. controlled pore glass (CPG) and macroporous polystyrene (MPPS). A preferred solid phase is a solid phase having a plurality of free amino groups.

Nucleosides or nucleoside analogs, nucleoside mono-, -di- and -triphosphates or mono-, di- and triphosphates of nucleoside analogs, and oligonucleosides may, for example, also be used as a nucleophile.

In the following, the invention is described in more detail by means of examples.

### EXAMPLE 1

### General Synthesis Scheme for 5′-Modified Oligonucleosides

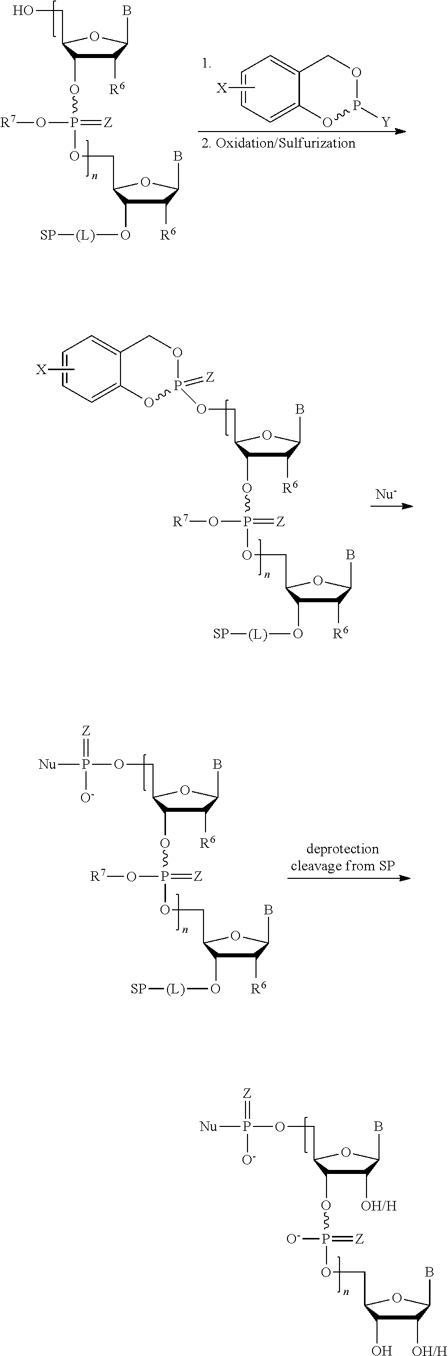

A general scheme for the synthesis of 5′-modified oligonucleosides is depicted above. B, X, Y, Z, (L), SP and R⁶ are defined as above. R⁷ is a protecting group, e.g. 2-cyanoethyl. Nu⁻ may be any nucleoside or nucleotide or phosphate or pyrophosphate, n is an integer≧0, e.g. 25.

In case of the preparation of an oligonucleoside, for example, a nucleoside (or di-, tri- or oligonucleoside, as the case may be) is bound, preferably via a linker L, e.g. via the 3′ C atom of the sugar component to a solid phase, e.g. controlled pore glass, by a method known in the art. A cycloSal compound according to formula II is reacted with the unprotected oxygen at the 5′ C atom of the sugar component of the immobilized nucleotide and the resulting compound is oxidized or sulfurized resulting in the corresponding phosphotriester, where Z may be O or Z. Sub sequently a nucleoside is reacted as nucleophile Nu⁻ with the immobilized cycloSal derivative, yielding a dinucleoside (or elongated oligonucleoside). The reactions may be repeated until an oligonucleoside of the desired length is received. Subsequently, the immobilized oligonucleoside may be deprotected and released from the solid phase SP.

### EXAMPLE 1.2

### Synthesis of pppT₇

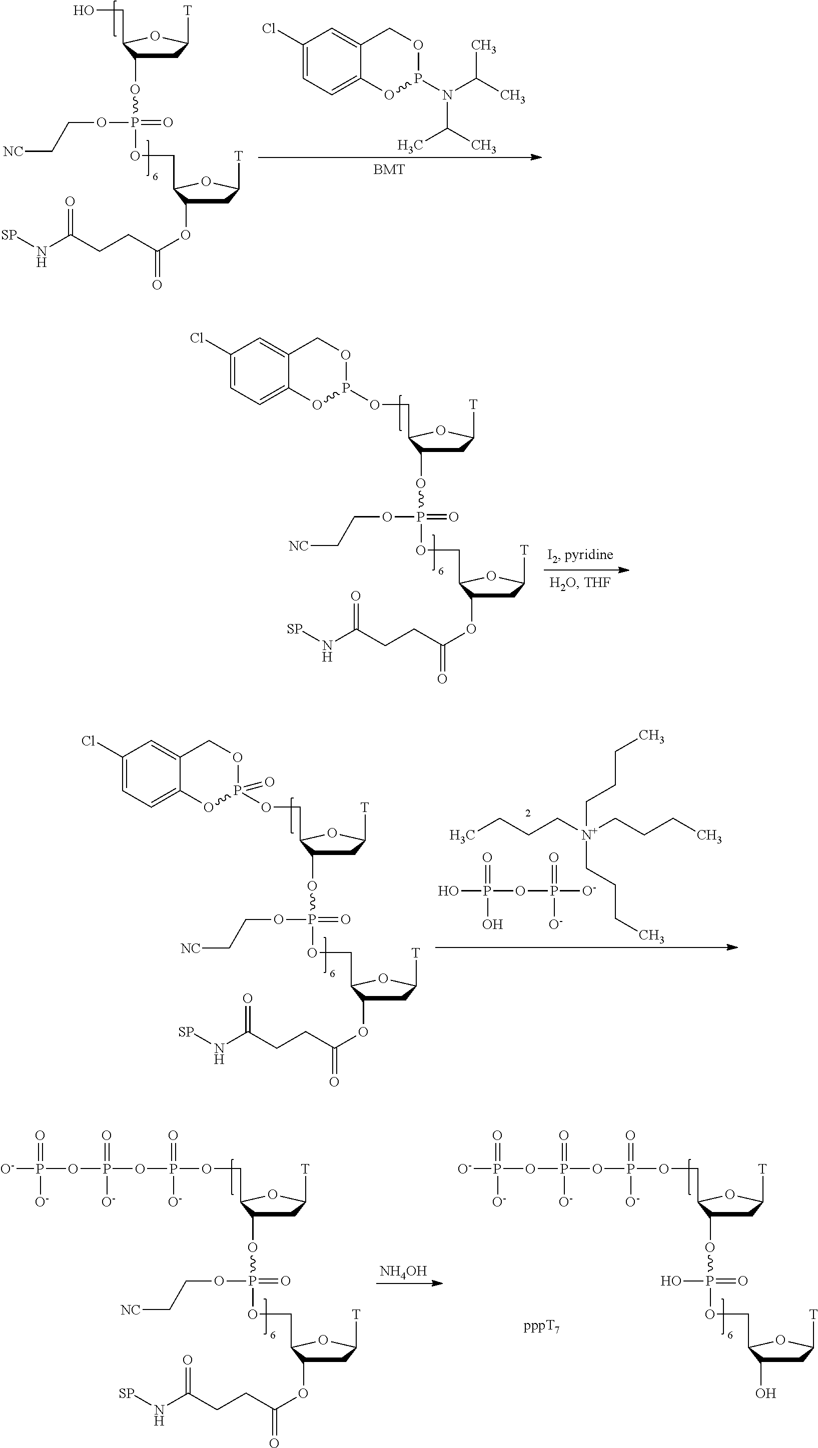

### 1.2.1 CycloSal Phosphoramidite Synthesis (3 Steps)

### 1.2.1.1 Synthesis of 5-Chlorsaligenol

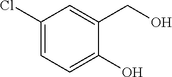

To a suspension of 1.52 g (40.1 mmol) LiAlH₄ in 50 mL dry diethylether 4.50 g (26.1 mmol) 5-chlorosalicylic acid in 50 mL dry diethylether was added dropwise at room temperature. The solution was refluxed for 45 minutes and subsequently cooled down to 0° C. in an ice bath. 50 mL water as well as 100 mL 10% H₂SO₄ was added dropwise under stirring. The solution was extracted three times with diethylether and washed with water. The combined organic layers were dried with Na₂SO₄ and filtered. The filtrate was concentrated in vacuo and the residue recrystallized in chloroform. The product is a crystalline solid (yield: 3.31 g, 80%).

### 1.2.1.2 Synthesis of 5-Chlorsaligenylchlorophosphite

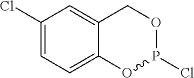 [SMILES: Clc1ccc2c(c1)COP(Cl)O2]

2.45 g (15.4 mmol) 5-Chlorsaligenol (s. 1.2.1.1) were coevaporated three times with dry acetonirile and solved in 75 mL dry diethylether. After cooling the reaction mixture down to −20° C. 2.86 mL (35.4 mmol) PCl₃ was added dropwise. The solution was stirred for 10 minutes, then 2.86 mL (35.4 mmol) dry pyridine diluted in 10 mL dry diethylether was added dropwise over 2 hours. The reaction mixture was stirred for another 2 hours at room temperature and stored at −26° C. over night. After filtration and evaporation the crude product was purified by “Kugelrohr” distillation. The product was obtained as a colorless oil (yield: 2.17 g, 63%).

### 1.2.1.3 Synthesis of 5-Chlorosaligenyl-N,N-diisopropylphosphoramite

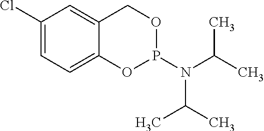 [SMILES: CC(C)N(C(C)C)P1OCc2cc(Cl)ccc2O1]

To a solution of 1.92 g (8.61 mmol) 5-Chlorsaligenyl-chlorophosphite (s. 1.2.1.2) in 50 mL dry diethylether 2.66 mL (18.9 mmol) dry diisopropylamine was added dropwise and the reaction mixture stirred for 1-2 hours. The solution was filtered and evaporated. After adding hexane and diisopropylamine in the ratio 9:1 (v/v) the crude product was purified by silica filtration. After evaporation to dryness the product was obtained as a white solid (yield: 2.16 g, 87%).

### 1.2.2 Preparation of Bis(Tetrabutylammonium)Dihydrogen Pyrophosphate

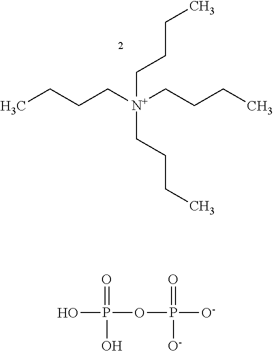 [SMILES: CCCC[N+](CCCC)(CCCC)CCCC ; O=P([O-])([O-])OP(=O)(O)O]

Tetrasodium pyrophosphate decahydrate (2.62 g, 5.87 mmol) was dissolved in 60 mL Milli-Q water and eluted through a column filled with 200 g wet DOWEX 50WX8 (50-100 mesh), H⁺ form. The corresponding diphosphoric acid was collected in a flask and 7.60 g (11.72 mmol) tetra-n-butylammonium hydroxide, 40% w/w aqueous solution, added dropwise while strirring in an ice bath. After lyophilization, grinding and drying in vacuo bis(tetrabutylammonium)dihydrogen pyrophosphate was obtained as a white powder (yield: 2.79 g, 72%). A 0.45 M solution in dry dimethylformamide was prepared by diluting 0.3 g per 1 mL dimethylformamide.

### 1.2.3 DNA Synthesizer Steps for pppT₇ Synthesis

Synthesis of the 2′-deoxyoligonucleotide T₇ was done on a DNA synthesizer after the well-established phosphoramidite method with the following cycle conditions:
1. Detritylation: 10×3% DCA (dichloroacetic acid) in DCM (dichloromethane) for 10 seconds.
2. Coupling: 3×0.1 M phosphoramidite with 0.3 M BMT (5-benzylthio-1H-tetrazole) in MeCN for 25, 45 and 20 seconds.
3. Oxidation: 1×0.02 M I₂, H₂O, pyridine in THF for 60 seconds.
4. Capping: 2×5% Phenoxyacetic anhydride in pyridine and THF for 25 seconds.

Once the DMT-on form of T₇ was synthesized the next three steps were executed:
1. Detritylation: 10×3% DCA in DCM for 10 seconds.
2. Coupling: 3×0.1 M cycloSal-phosphoramidite with 0.3 M BMT in MeCN for 20, 40 and 40 seconds.
3. Oxidation: 1×0.02 M I₂, H₂O, pyridine in THF for 20 seconds.

The subsequent triphosphorylation reaction was done by pushing 0.45 M bis(tetrabutyl-ammonium)dihydrogen pyrophosphate in DMF (dimethylformamide) through the synthesis column and let it left to react for 180 seconds. After this step the solution was pushed four more times through the column, 30 minutes between pushes. The total phosphorylation time was 2 hours. The supported triphosphorylated oligonucleotide was washed with dry DMF, MeCN and dried under argon flow.

### 1.2.4 Deprotection and Purification

Deprotection was done manually with two syringes: 1.5 mL of 33% NH₃ in water was pushed through the column and left to react for 1 hour. The solution was collected in a flask and another 1 mL NH₄OH was pushed through the column and left to react for 30 minutes. This step was repeated one last time with 0.5 mL NH₄OH. In total the oligonucleotide was deprotected with 3 mL NH₄OH in 2 hours. After filtration the solvent was evaporated and the crude triphosphorylated oligonucleotide purified by IEX-HPLC with 20 mM sodium phosphate buffer (pH=7.2) and a 0.5 M NaCl gradient in 40 minutes with a flow rate of 1 mL/min.

### EXAMPLE 2

### Synthesis of Thymidine 5′ Triphosphate (pppT)

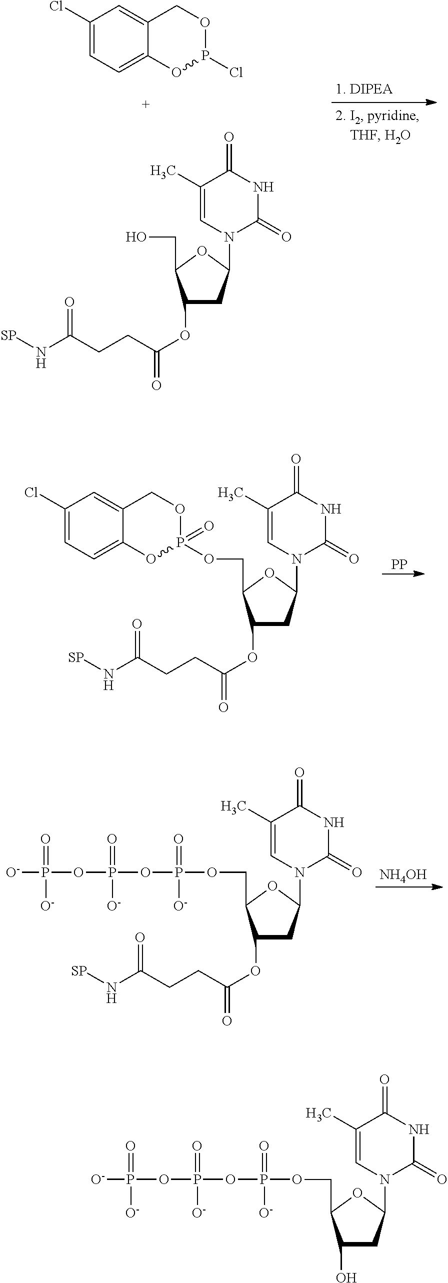

15 mg (16 μmol) thymidine, attached via a succinyl linker to polystyrene (1% DVB) was filled in a synthesis column. 11 μL (65 μmol) of dry diisopropylethylamine and 15 mg (67 μmol) 5-chlorsaligenylchlorophosphite (s. 1.2.1.2) were dissolved in 1 mL dry DMF. The solution was pushed back and forth through the synthesis column for 1 hour. The solution was removed and washed with dry DMF and MeCN. The corresponding phosphite was oxidized with 0.1 M oxidizer solution (I₂, H₂O, pyridine in THF), washed with dry DMF and MeCN and dried under argon flow. The polystyrene bound nucleotide was transferred to an eppendorf vial and phosphorylated with 1 mL of the previously described 0.45 M solution of bis(tetrabutylammonium)dihydrogen pyrophosphate in dimethylformamide (s. 1.2.2). It was mixed for 17 hours at room temperature. After washing with dry DMF and MeCN the product was cleaved with NH₄OH at 55° C. for 2 hours.

1. A method for the solid-phase based synthesis of a compound of the general formula I 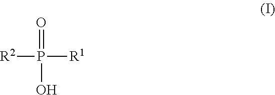
or a salt thereof, wherein
R¹ is a nucleoside, nucleotide, polynucleoside, polynucleotide or an analog thereof,
R² is an organic compound or phosphate or pyrophosphate, or a residue thereof, the method comprising the steps of:
a) immobilizing a compound being or comprising R¹ directly or via a linker L on a solid phase SP,
b) coupling to the immobilized compound a substituted or unsubstituted compound of the general formula II 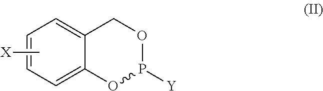
X being H, an electron acceptor or an electron acceptor precursor and Y being halogen or —NR³R⁴, wherein R³ and R⁴ are, independently, substituted or unsubstituted alkyl or substituted or unsubstituted aryl, and wherein the compound II may be substituted one or more times with X, and oxidizing or sulfurizing the resulting compound to obtain an immobilized compound according to the general formula III 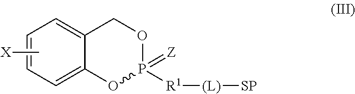
R¹ and X being as defined above, Z being O or S, SP being the solid phase and (L) being the optional linker, and
c) reacting the compound III with a nucleophile being or comprising R².

2. The method according to claim 1, wherein R¹ is selected from the group consisting of oligonucleoside, oligonucleotide, oligonucleoside analog, oligonucleotide analog, adenosine, guanosine, cytidine, thymidine, uridine, deoxyadenosine, deoxyguanosine, inosine, deoxycytidine, deoxyuridine, deoxythymidine, 2-thiocytidine, N⁴-acetyl-cytidine, 2′-O-methyl-cytidine, 3-methyl-cytidine, 5-methyl-cytidine, 2-thiouridine, pseudouridine, dihydrouridine, 5-(carboxyhydroxymethyl)-uridine, 5-carboxymethyl-aminomethyl-uridine, 5-methylaminomethyl-uridine, 5-methoxy-carbonylmethyl-uridine, 5-methoxy-uridine, ribothymidine, 1-methyl-adenosine, 2-methyl-adenosine, N⁶-methyl-adenosine, inosine, 1-methyl-inosine, guanosine, N²-dimethyl-guanosine, N²-methyl-guanosine, 7-methyl-guanosine and 2′-O-methylguanosine.

3. The method according to claim 1, wherein the solid phase or the linker are covalently bound to an oxygen atom of a sugar component of R¹, preferably an oxygen atom bound to the 2′- or 3′ C atom of the sugar component, or to an oxygen atom of a component analogous to a sugar component of R¹, and wherein the residue of formula IIa 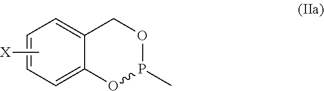
is linked to a different oxygen atom of the same or another sugar component of R¹, preferably an oxygen atom bound to the 5′ C atom of said sugar component, or to a different oxygen atom of the same or another component analogous to a sugar component of R¹.

4. The method according to claim 3, wherein compound III is a compound according to formula IIIa 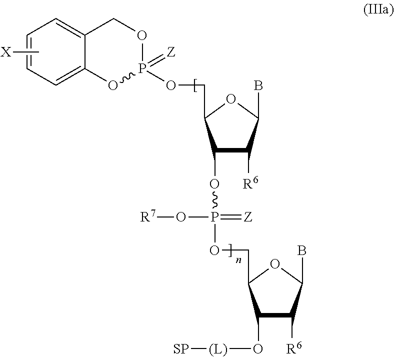
wherein X, Z, SP and (L) are defined as above, B is, independently for each occurrence, a heterocycle, preferably a nitrogen containing heterocycle, especially preferred a nucleobase, R⁶ is, independently for each occurrence, H or OPG, PG representing a protecting group, R⁷ is, independently for each occurrence, H or OPG, PG representing a protecting group, and n is an integer≧0.

5. The method according to claim 4, wherein Z is O or S, preferably O, and B is one of the nucleobases guanine, adenine, cytosine, thymine or uracil.

6. The method according to claim 1, wherein the solid phase or the linker is covalently bound to a nitrogen atom of a base component of the nucleoside, nucleotide, polynucleoside or polynucleotide, or to a nitrogen atom of a component analogous to a base component of the nucleoside analog, nucleotide analog, polynucleoside analog or polynucleotide analog of R¹.

7. The method according to claim 1, wherein X, in case of multiple substituents X independently from each other, is selected from the group consisting of H, MeSO₂—, ketone, formyl, ester, —C═O, —CN, —COOH, —NO₂ and halogen.

8. The method according to claim 1, wherein the nucleophile is selected from the group consisting of phosphate, pyrophosphate, glycosyl phosphate, nucleoside, nucleoside monophosphate, nucleoside diphosphate, nucleoside triphosphate, nucleoside analog, nucleoside monophosphate analog, nucleoside diphosphate analog, nucleoside triphosphate analog, α-deprotonated glycosyl, deprotonated mono- or oligosaccharide, amines, amino acids, lipids, steroids, or salts thereof.

9. The method according to claim 1, wherein steps a, b and c are carried out repeatedly.

10. The method according to claim 1, comprising the further step(s) of
d) deprotecting compound III and/or cleaving the residue R¹ from the linker or the solid phase.

11. The method according to claim 1, wherein the method is carried out under inert gas atmosphere, preferably under nitrogen or argon gas.
